(19) United States
(12) Patent Application Publication
(10) Pub. No.: US 2015/0045333 A1
(43) Pub. Date: Feb. 12, 2015

(54) LIQUID COMPOSITIONS COMPRISING VITAMIN D AND USES THEREOF

(71) Applicant: FIT-BioCeuticals Pty Ltd, Alexandria (AU)

(72) Inventors: Evan Hayes, Darlinghurst (AU); Sean Michael Hall, Sydney (AU); Melissa Luke, Stanmore (AU)

(73) Assignee: FIT-BioCeuticals Pty Ltd.

(21) Appl. No.: 14/521,055

(22) Filed: Oct. 22, 2014

Related U.S. Application Data
(63) Continuation of application No. 13/513,553, filed on Sep. 17, 2012, now abandoned.

(30) Foreign Application Priority Data
Dec. 2, 2009 (AU) 2009905881

(51) Int. Cl. A61K 9/107; A23D 9/007; A61K 31/592; A61K 31/593; A61K 47/12

(52) U.S. Cl. CPC A61K 9/107; A61K 31/593; A61K 47/12; A61K 31/592; A23D 9/007
USPC 514/167

(57) ABSTRACT

The present invention relates to a liquid composition comprising vitamin D, or a salt thereof. Specific embodiments employ 5000 IU to 250,000 IU, and include sorbate and benzoate as a preserving agent.

### CROSS REFERENCE TO RELATED APPLICATIONS

This is a continuation of co-pending U.S. patent application Ser. No. 13/513,553, filed Jun. 1, 2012, which is the U.S. National Stage of International Application No. PCT/AU2010/001626, filed Dec. 2, 2010, which was published in English under PCT Article 21(2), which in turn claims priority to Australian Provisional Patent Application No. 2009905881 filed Dec. 2, 2009, the contents of which are hereby incorporated by reference.

### TECHNICAL FIELD

The present invention relates to liquid compositions comprising vitamin D, methods for the preparation thereof, and also to the use of such compositions in the treatment of conditions associated with vitamin D deficiency and to supplement the amount of vitamin D in the body of a subject.

### BACKGROUND OF THE INVENTION

The term “Vitamin D” encompasses a group of fat-soluble prohormones. The two principal forms of vitamin D are vitamin D₂ (or ergocalciferol) and vitamin D₃ (or cholecalciferol). Cholecalciferol is produced endogenously in human skin when 7-dehydrocholesterol is irradiated with light having a wavelength between 270 and 300 nm. It is accepted that adequate amounts of vitamin D₃ can only be produced after sun exposure of certain parts of the body (i.e. face, arms, hands or back) for a period of 10 to 15 minutes at least twice a week in the absence of sunscreen.

A balanced level of vitamin D has long been recognised as essential to health. Vitamin D appears to increase the efficiency of the intestines to absorb calcium and also mobilizes calcium from bone tissue when required. Vitamin D deficiency can result from a number of factors including: inadequate intake coupled with inadequate sunlight (UVB) exposure, disorders that limit its absorption from the GI tract, conditions that impair conversion of vitamin D into active metabolites (such as liver or kidney disorders) and body characteristics, such as skin colour and body fat.

Vitamin D deficiency results in impaired bone mineralisation and leads to bone softening diseases such as rickets and osteoporosis, and may also be a contributing factor to high blood pressure, tuberculosis, cancer, heart disease, stroke, periodontal disease, MS, seasonal affective disorder and memory loss. Vitamin D deficiency is commonly observed in children, the aged and those of lower socio-economic status.

Current research suggests that vitamin D deficiency requires treatment at a considerably higher level than the current Australian Government mandated limit of 1000 IU per day (self prescription). This would require the administration of many solid dosage forms (for example gelatin capsules) each day.

Certain individuals suffering from the above conditions and children in general are unable and/or unwilling to consume multiple solid dosage forms on a daily basis, and many other individuals find that in order to consume an effective amount of vitamin D, the number of dosage forms required (for example tablets and gel capsules) is plainly inconvenient.

Against this background, the present inventors have developed concentrated liquid compositions comprising vitamin D that are capable of providing a high dose of vitamin D in a relatively small total liquid amount.

### SUMMARY OF THE INVENTION

In a first aspect, the present invention provides a liquid composition comprising vitamin D, or a salt thereof, in an amount between about 1000 IU and about 500,000 IU per mL of the composition.

The composition may comprise vitamin D, or a salt thereof, in an amount between about 5000 IU and about 250,000 IU per mL of the composition. In an alternative embodiment, the composition may comprise vitamin D, or a salt thereof, in an amount between about 5000 IU and about 100,000 IU per mL of the composition, or in an amount between about 5000 IU and 50,000 IU per mL of the composition, or in an amount between about 10,000 IU and about 50,000 IU per mL of the composition.

The vitamin D may be present in the composition as 1α,25-dihydroxy vitamin D₃, or an analogue or derivative thereof.

The vitamin D may be present in the composition as a precursor of 1α,25-dihydroxy vitamin D₃ or as a precursor of an analogue or derivative of 1α,25-dihydroxy vitamin D₃, wherein the precursor of 1α,25-dihydroxy vitamin D₃ or the precursor of an analogue or derivative of 1α,25-dihydroxy vitamin D₃ is metabolised in vivo to provide 1α,25-dihydroxy vitamin D₃ or an analogue or derivative of 1α,25-dihydroxy vitamin D₃.

The precursor may be cholecalciferol.

The vitamin D may be present in the composition as ergocalciferol, or a metabolite, analogue or derivative thereof.

The composition may further comprise an oil, for example an edible oil such as a vegetable oil.

The vegetable oil may be rice bran oil.

The oil may be present in an amount between about 100 and 600 mg/mL of the composition, or in an amount between 200 and 400 mg/mL of the composition.

The composition may further comprise one or more surfactants.

The surfactant may be a gum, for example a natural gum.

The surfactant may be present in an amount between about 30 and 150 mg/mL of the composition, or in an amount between 60 and 120 mg/mL of the composition.

The composition may further comprise one or more preservatives.

The preservative may be selected from the group consisting of: sorbates and benzoates, for example sodium benzoate and potassium sorbate.

In one embodiment, the preservative is a preservative system comprising a combination of one or more benzoates and one or more sorbates.

The ratio by mass of the amount of benzoate present in the composition to the amount of sorbate present in the composition may be between about 1:1 and about 5:1.

The composition may be in the form of an emulsion.

The emulsion may be an oil-in-water emulsion or a water-in-oil emulsion.

The emulsion may be a microemulsion.

The composition may further comprise flavours and/or colours so as to enhance its palatability and/or visual appearance.

The composition may be a composition intended for oral administration.

In an embodiment of the first aspect, the present invention provides a liquid emulsion composition comprising vitamin D in an amount between about 10,000 IU and 250,000 IU per mL, water, an edible oil, a natural gum, sorbate and benzoate.

The edible oil may be present in an amount between about 100 and 600 mg/mL, the natural gum may be present in an amount between about 30 and 180 mg/mL, the sorbate may be present in an amount between about 2 and 90 mg/mL, the benzoate may be present in an amount between about 5 and 170 mg/mL, with water making up the remainder of the composition.

The edible oil may be present in an amount between about 200 and 400 mg/mL, the natural gum may be present in an amount between about 60 and 120 mg/mL, the sorbate may be present in an amount between about 5 and 70 mg/mL, the benzoate may be present in an amount between about 10 and 140 mg/mL, with water making up the remainder of the composition.

In a second aspect, the present invention provides a method for preparing a liquid composition of the first; aspect, the method comprising:
a) admixing a surfactant and water to provide a surfactant/water mixture;
b) admixing vitamin D, or a salt thereof, and an oil to provide a vitamin D/oil mixture;
c) admixing the surfactant/water mixture and the vitamin D/oil mixture;
d) adding one or more preservatives to the mixture obtained following step c); and
e) adding water to the mixture obtained following step d).

The surfactant, oil and preservative may be as defined in the first aspect and as defined in the detailed description that follows.

Step a) may comprise agitating the surfactant/water mixture.

Step b) may comprise agitating the vitamin D/oil mixture.

Step c) may comprise admixing the vitamin D/oil mixture and the surfactant/water mixture under homogenisation conditions.

Step d) may comprise adding one or more preservatives, and water to the mixture obtained following step c).

The method may further comprise agitating the mixture obtained following step e) until a homogeneous liquid is obtained.

The method may further comprise adding one or more preservatives as part of step b) and/or step a).

In a third aspect, the present invention provides a method for the prevention and/or treatment of a disease or condition in a subject associated with a deficiency of vitamin D, said method comprising administration to the subject of a therapeutically effective amount of a composition of the first aspect.

The disease or condition may be selected from the group consisting of: vitamin D deficiency syndrome, rickets, osteoporosis, hypertension, chronic fatigue, chronic pain, autoimmune diseases, hype arathyroidism, high blood pressure, tuberculosis, cancer, depression, heart disease, stroke, periodontal disease, MS, seasonal affective disorder and memory loss.

In a fourth aspect, the present invention provides a method for supplementing the amount of vitamin D in the body of a subject, said method comprising administration to the subject of a composition of the first aspect.

The subject may be an animal, for example a human.

These and other aspects of the invention will become evident from the description and claims which follow.

### DEFINITIONS

The following are some definitions that may be helpful in understanding the description of the present invention. These are intended as general definitions and should in no way limit the scope of the present invention to those terms alone, but are put forth for a better understanding of the following description.

Throughout this specification, unless the context requires otherwise, the word “comprise”, or variations such as “comprises” or” comprising”, will be understood to imply the inclusion of a stated step or element or integer or group of steps or elements or integers, but not the exclusion of any other step or element or integer or group of elements or integers. Thus, in the context of this specification, the term “comprising” means “including principally, but not necessarily solely”.

In the context of this specification, the term “about” is understood to refer to a range of numbers that a person of skill in the art would consider equivalent to the recited value in the context of achieving the same function or result.

In the context of this specification, the terms “a” and “an” are used herein to refer to one or to more than one (i.e. to at least one) of the grammatical object of the article. By way of example, “an element” means one element or more than one element.

In the context of this specification, the term “vitamin D” is to be given its broadest construction and is understood to include all vitamers and precursors thereof. The term includes all biologically active forms of vitamin D (for example calcitriol) as well as analogues, derivatives and precursors thereof. The term “vitamin D” also includes all compounds which exhibit biological properties similar to those of vitamin D, in particular the properties of transactivation of the vitamin D response elements (VDRE), such as an agonist or antagonist activity towards receptors for vitamin D.

In the context of this specification, the terms “treatment” and “treating” refer to any and all uses which remedy a condition, disease, disorder or symptoms thereof, or otherwise prevent, hinder or reverse the progression of a condition, disease, disorder or symptoms thereof, in any way whatsoever. Treatment may be for a defined period of time, or provided on an ongoing basis depending on the particular circumstances of any given individual.

In the context of this specification, the terms “prevent” and “prevention” refer to any and all uses which prevent the establishment or onset of a condition, disease, disorder or symptoms thereof in any way whatsoever.

In the context of this specification, the term “therapeutically effective amount” includes within its meaning a non-toxic amount of a composition sufficient to provide the desired therapeutic effect. The exact amount will vary from subject to subject depending on the age of the subject, their general health, the severity of the disorder being treated and the mode of administration. It is therefore not possible to specify an exact “therapeutically effective amount”, however one skilled in the art would be capable of determining such an amount by routine trial and experimentation.

In the context of this specification, the term “salts thereof is understood to include acid addition salts, anionic salts and zwitterionic salts, and in particular pharmaceutically acceptable salts.

In the context of this specification, the term “pharmaceutically acceptable” means that the compound to which it refers is suitable for use in contact with tissues of the body without undue toxicity, incompatibility, instability, irritation, allergic response and the like, commensurate with a reasonable benefit/risk ratio. Pharmaceutically acceptable salts include salts of acidic or basic groups present in compounds. Examples of salts include but are not limited to, those formed from: acetic, ascorbic, aspartic, benzoic, benzenesulfonic, citric, cinnamic, ethanesulfonic, fumaric, glutamic, glutaric, gluconic, hydrochloric, hydrobromic, lactic, maleic, malic, methanesulfonic, naphthoic, hydroxynaphthoic, naphthalenesulfonic, naphthalenedisulfonic, naphthaleneacrylic, oleic, oxalic, oxaloacetic, phosphoric, pyruvic, p-toluenesulfonic, tartaric, trifluoroacetic, triphenylacetic, tricarballylic, salicylic, sulphuric, sufamic, sulfamlic and succinic acid. Pharmaceutically acceptable salts also include alkali or alkaline earth metal salts of acidic groups and/or hydroxy groups, and also any other non-toxic metal salts.

In the context of this specification the term “stable emulsion” refers to an emulsion which does not separate into oil and water phases upon standing.

### DETAILED DESCRIPTION OF THE INVENTION

In a first aspect, the present invention provides a liquid composition comprising vitamin D, or a salt thereof, in. an amount between about 1000 IU and about 500,000 IU per mL of the composition.

The most abundant form of vitamin D is vitamin D₃, or cholecalciferol. Cholecalciferol is produced endogenously in human skin when 7-dehydrocholesterol is irradiated with light having a wavelength between 270 and 300 nm Metabolism of cholecalciferol occurs in the liver to produce 25-hydroxy vitamin D₃ (calcidiol) which is a major form of Vitamin D circulating in the blood. 25-hydroxy vitamin D₃ is then converted by the kidney to produce two principal dihydroxylated metabolites, namely 1α,25-dihydroxy vitamin D₃ (calcitriol) and 24,25-dihydroxy vitamin D₃.

1α,25-dihydroxy vitamin D₃ is the most biologically active naturally occurring form of vitamin D. However it will be appreciated by persons skilled in the art that the present invention is not limited to compositions and methods comprising the administration of 1α,25-dihydroxy vitamin D₃. Any suitable precursor, previtamin, derivative or analogue thereof may be used. For example, the vitamin D included in the compositions may be a hydroxylated metabolite or other metabolic product of vitamin D₃. Suitable vitamin D₃ analogues (deltanoids) are described, for example, in Guyton, K. Z. et al. (2003) Nutrition Reviews 61:227-238, and Gaschott et al. (2002) Biophys Biochem Research Comm 290:504-509, the disclosures of which are incorporated herein by reference. Suitable analogues include, but are not limited to: 22-oxacalcitriol (OCT), calcipotriol (MC903), 1α,25-dihydroxy-22,24-diene-24,26,27-trihomo vitamin D_{3 (EB} 1089), 1α-hydroxy vitamin D₅,16-ene-23-yne-26,27-hexafluoro-1α,25 dihydroxy vitamin D₃ (Ro 24-5531), 16-ene-23-yne-19-nor-26,27-hexafiuoro-1α,25 dihydroxy vitamin D₃ (Ro 25-6760), 16,23E-diene-19-nor-26,27-hexafluoro-1α,25 dihydroxy vitamin D₃ (Ro 25-9022), 22E,24E-diene-24,26a,27a-trihomo-1α,25 dihydroxy vitamin D₃ (Leo EB-1089), 20-epi-22-oxa-26a,27a-bishomo-1α,25 dihydroxy vitamin D₃ (Leo KH-1060), iβ-hydroxymethyl-3-epi-16-ene-24,24-difluoro-26a,27a-bishomo-25 dihydroxy vitamin D₃ (Hopkins-QW-1624F₂-2), 24R,25 dihydroxyvitamin D₃, TX 522, TX 527, 20-epi-1,25-dihydroxy vitamin D₃ and ZK 156718. Suitable precursors include, for example, any and all compounds which are metabolised in vivo to provide biologically active forms of vitamin D₃ (or analogues or derivatives thereof), such as cholecalciferol. Other suitable precursors, derivatives and analogues of biologically active forms of vitamin D₃ will be known to those skilled in the art.

The vitamin D present in the compositions may also be in the form of vitamin D₂ (ergocalciferol), or metabolites, analogues or derivatives thereof. Examples of suitable vitamin D₂ compounds include, but are not limited to: ergocalciferol and metabolites thereof (for example hydroxylated metabolites such as 25-hydroxy vitamin D₂ and 1,25-dihydroxy vitamin D₂), 19-nor-1,25-dihydroxy vitamin D₂ and dihydrotachysterol.

The vitamin D present in the compositions may be in the form of a salt, for example a pharmaceutically acceptable salt.

The vitamin D in the compositions may be present in an amount between about 5000 IU and about 250,000 IU, or in an amount between about 5000 IU and about 200,000 IU, or in an amount between about 5000 IU and about 150,000 IU, or in an amount between about 5000 IU and about 100,000 IU, or in an amount between about 5000 IU and about 75,000 IU, or in an amount between about 5000 IU and about 50,000 IU, or in an amount between about 10,000 IU and about 250,000 IU, or in an amount between about 20,000 IU and about 250,000 IU, or in an amount between about 50,000 IU and about 250,000 IU, or in an amount between about 50,000 IU and about 200,000 IU, or in an amount between about 50,000 IU and about 150,000 IU, or in an amount between about 50,000 IU and about 100,000 IU, or in an amount between 20,000 IU and about 200,000 IU or in an amount between 20,000 IU and about 150,000 IU, or in an amount between about 20,000 IU and about 100,000 IU. The above amounts are per mL of the composition.

In one embodiment, the composition is in the form of an emulsion. The emulsion may be, for example, a water-in-oil emulsion or an oil-in-water emulsion. The emulsion may be stable when stored at a temperature below 30° C. in the absence of light for a period of up to 3 months, 6 months, 9 months, 1 year, 2 years, 3 years, 4 years or 5 years.

The composition may further comprise one or more oils. Suitable oils include any edible oils, for example rice bran oil, corn oil, soybean oil, canola oil, palm oil, rapeseed oil, sunflower oil, peanut oil, coconut oil, olive oil, safflower oil, linseed oil, grapeseed oil, sesame oil, hazelnut oil, cottonseed oil and the like. The edible oil may be a vegetable oil or a nut oil. The oil may be an oil that has a taste that is capable of being attenuated or masked sufficiently such that the composition has a pleasant taste.

The composition may further comprise one or more surfactants. The surfactant may be any non-toxic surfactant which is suitable for consumption by humans (i.e. any edible surfactant). The surfactant may be an anionic, non-ionic or cationic surfactant. Suitable surfactants include, but are not limited to: fatty acids and esters thereof derived from natural oils (such as oleic acid, stearic acid, palmitic acid and esters thereof), ethylene oxide derivatives, ethoxylated linear alcohols, ethoxylated alkyl phenols, amine and amide derivatives, alkylpolyglucosides, ethylene oxide/propylene oxide copolymers, polyalcohols, ethoxylated polyalcohols and mercaptan derivatives.

In one embodiment, the surfactant is a gum, for example a natural gum or a cellulosic gum. Suitable natural gums include, but are not limited to: gum arabic, agar, alginic acid, glucan, carrageenan, chicle gum, dannar gum, gellan gum, glucomannan, guar gum, ghatti, tragacanth, pectin, karaya gum, locust bean gum, mastic gum, alginate, spruce gum, tara gum and xanthan. Cellulosic gums include but are not limited to: methylcellulose, carboxymethylcellulose, hydroxyethylcellulose, hydroxypropylcellulose, hydroxyethylmethylcellulose and hydroxypropylmethylcellulose.

The composition may further comprise one or more preservatives. Preservatives for liquid compositions are well known to those skilled in the art and include, but are not limited to: ethanol, glycerine, lemongrass, rosemary, benzoate (for example sodium benzoate), sorbate (for example potassium sorbate), salts of EDTA, parabens (for example methyl, ethyl, propyl and butyl p-hydroxybenzoic acid esters, or mixtures thereof) and mixtures thereof. In one embodiment, the preservative is a preservative system comprising sodium benzoate and potassium sorbate. The ratio by mass of the amount of benzoate present in the composition to the amount of sorbate present in the composition may be between about 1:1 and about 5:1, or between about 1:1 and about 4:1, or between about 1:1 and about 3:1, or between about 1.4:1 and about 2.5:1.

The composition may further comprise flavours and/or colours so as to enhance its palatability and/or visual appearance. Suitable flavouring agents and colouring agents are well known to those skilled in the art. The flavouring agent may be a natural or artificial flavouring agent, including an essence, an extract, a flavour oil or combinations thereof. Exemplary flavours include, but are not limited to: honey flavour, raspberry flavour, strawberry flavour, blueberry flavour, blackberry flavour, grape flavour, peach flavour, apricot flavour, watermelon flavour, melon flavour, fruit punch flavour, cranberry flavour, mango flavour, banana flavour, citrus flavour, orange flavour, lemon flavour, grapefruit flavour, cherry flavour, vanilla flavour, mocha flavour, caramel flavour, butter rum flavour, chocolate flavour, marshmallow flavour, coffee flavour, coconut flavour and butterscotch flavour.

The compositions of the present invention are not limited to any particular pH. The composition may have a pH in the range of about 3 to about 7, or in the range of about 3.5 to about 6.5. It may be advantageous for the composition to have a pH in the range of about 4.5 to about 5.0 so as to inhibit or minimise agglomeration and settling of gum ingredients within the composition.

In one embodiment, the composition is a composition that is intended for oral administration, and hence may include non-toxic carriers, diluents and/or excipients in addition to those described above.

Based on the concentration of vitamin D in the compositions of the present invention, it is possible to administer a large dose of vitamin D (for example 10,000 IU) to a subject in a relatively small total liquid amount (for example as little as 1 mL). This is particularly advantageous in light of the suggestions by current research that humans should significantly increase their intake of vitamin D in order to potentially avoid serious health consequences. The compositions of the present invention also provide significant advantages over currently available solid dosage forms. For example, in order to consume 10,000 IU of vitamin D on a daily basis it is necessary to consume many solid dosage forms throughout the day. This is not only inconvenient, but in the case of children and individuals having certain conditions associated with vitamin D deficiency, not always possible. In contrast, by administering the compositions of the present invention, one is able to consume 10,000 IU of vitamin D by taking only 1 mL total solution.

In an embodiment of the first aspect, the composition is an emulsion composition intended for oral administration comprising: vitamin D in an amount between 5000 IU and 250,000 IU per mL, water, an edible oil, a surfactant, sorbate and benzoate.

In another embodiment of the first aspect, the composition is an emulsion composition intended for oral administration comprising: vitamin D in an amount between 5000 IU and 100,000 IU per mL, water, an edible oil, a surfactant, sorbate and benzoate.

In another embodiment of the first aspect, the composition is an emulsion composition intended for oral administration comprising: vitamin D in an amount between 10,000 IU and 50,000 IU per mL, water, an edible oil, a surfactant, sorbate and benzoate.

In another embodiment of the first aspect, the composition is an emulsion composition intended for oral administration comprising: vitamin P in an amount between 5000 IU and 250,000 IU per mL, water, an edible oil, a natural gum, sorbate and benzoate.

In another embodiment of the first aspect, the composition is an emulsion composition intended for oral administration comprising: vitamin P in an amount between 5000 IU and 250,000 IU per mL, water, a vegetable oil, a natural gum, sorbate and benzoate.

In a further embodiment of the first aspect, the composition is an emulsion composition intended for oral administration comprising: cholecalciferol in an amount between 5000 IU and 250,000 IU per mL, water, rice bran oil, a natural gum, sorbate and benzoate.

In still a further embodiment of the first aspect, the composition is an emulsion composition intended for oral administration comprising: cholecalciferol in an amount between 5000 IU and 250,000 IU per mL, water, rice bran oil, gum arabic, sorbate and benzoate.

In yet another embodiment of the first aspect, the composition is an emulsion composition intended for oral administration comprising: vitamin D in an amount between 5000 IU and 50,000 IU per mL, water, an edible oil, a natural gum, sorbate and benzoate.

In a further embodiment of the first aspect, the composition is an emulsion composition intended for oral administration comprising: vitamin P in an amount between 5000 IU and 50,000 IU per mL, water, a vegetable oil, a natural gum, sorbate and benzoate.

In another embodiment of the first aspect, the composition is an emulsion composition intended for oral administration comprising: cholecalciferol in an amount between 5000 IU and 50,000 IU per mL, water, rice bran oil, a natural gum, sorbate and benzoate.

In still a further embodiment of the first aspect, the composition is an emulsion composition intended for oral administration comprising: cholecalciferol in an amount between 5000 IU and 50,000 IU per mL, water, rice bran oil, gum arabic, sorbate and benzoate.

In yet another embodiment of the first aspect, the composition is an emulsion composition intended for oral administration comprising: vitamin D in an amount between 5000 IU and 250,000 IU per mL, an edible oil in an amount between about 200 and 400 mg/mL, a surfactant in an amount between about 60 and 120 mg/mL, sorbate in an amount between about 5 and 20 mg/mL, benzoate in an amount between about 10 and 30 mg/mL, with water making up the remainder of the composition.

In yet another embodiment of the. first aspect, the composition is an emulsion composition intended for oral administration comprising: vitamin D in an amount between 10,000 IU and 50,000 IU per mL, an edible oil in an amount between about 200 and 400 mg/mL, a surfactant in an amount between about 60 and 120 mg/mL, sorbate in an amount between about 5 and 20 mg/mL, benzoate in an amount between about 10 and 30 mg/mL, with water making up the remainder of the composition.

In yet another embodiment of the first aspect, the composition is an emulsion composition intended for oral administration comprising: vitamin D in an amount between 5000 IU and 250,000 IU per mL, an edible oil in an amount between about 200 and 400 mg/mL, a natural gum in an amount between about 60 and 120 mg/mL, sorbate in an amount between about 5 and 20 mg/mL, benzoate in an amount between about 10 and 30 mg/mL, with water making up the remainder of the composition.

In still a further embodiment of the first aspect, the composition is an emulsion composition intended for oral administration comprising: vitamin D in an amount between 5000 IU and 250,000 IU per mL, a vegetable oil in an amount between about 200 and 400 mg/mL, a natural gum in an amount between about 60 and 120 mg/mL, sorbate in an amount between about 5 and 20 mg/mL, benzoate in an amount between about 10 and 30 mg/mL, with water making up the remainder of the composition.

In another embodiment of the first aspect, the composition is an emulsion composition intended for oral administration comprising: cholecalciferol in an amount between 5000 IU and 250,000 IU per mL, rice bran oil in an amount between about 200 and 400 mg/mL, a natural gum in an amount between about 60 and 120 mg/mL, sorbate in an amount between about 5 and 20 mg/mL, benzoate in an amount between about 10 and 30 mg/mL, with water making up the remainder of the composition.

In a further embodiment of the first aspect, the composition is an emulsion composition intended for oral administration comprising: cholecalciferol in an amount between 10,000 IU and 100,000 IU per mL, rice bran oil in an amount between about 200 and 400 mg/mL, gum arabic in an amount between about 60 and 120 mg/mL, sorbate in an amount between about 5 and 20 mg/mL, benzoate in an amount between about 10 and 30 mg/mL, with water making up the remainder of the composition.

In yet another embodiment of the first aspect, the composition is an emulsion composition intended for oral administration comprising: vitamin D in an amount between 10,000 and 100,000 IU per mL, an edible oil in an amount between about 200 and 400 mg/mL, a natural gum in an amount between about 60 and 120 mg/mL, sorbate in an amount between about 5 and 20 mg/mL, benzoate in an amount between about 10 and 30 mg/mL, with water making up the remainder of the composition.

In still another embodiment of the first aspect, the composition is an emulsion composition intended for oral administration comprising: vitamin D in an amount between 10,000 IU and 100,000 IU per mL, a vegetable oil in an amount between about 200 and 400 mg/mL, a natural gum in an amount between about 60 and 120 mg/mL, sorbate in an amount between about 5 and 20 mg/mL, benzoate in an amount between about 10 and 30 mg/mL, with water making up the remainder of the composition.

In a further embodiment of the first aspect, the composition is an emulsion composition intended for oral administration comprising: cholecalciferol in an amount between 10,000 IU and 100,000 IU per mL, rice bran oil in an amount between about 200 and 400 mg/mL, a natural gum in an amount between about 60 and 120 mg/mL, sorbate in an amount between about 5 and 20 mg/mL, benzoate in an amount between about 10 and 30 mg/mL, with water making up the remainder of the composition.

In yet another embodiment of the first aspect, the composition is an emulsion composition intended for oral administration comprising: cholecalciferol in an amount between 10,000 IU and 100,000 IU per mL, rice bran oil in an amount between about 200 and 400 mg/mL, gum arabic in an amount between about 60 and 120 mg/mL, sorbate in an amount between about 5 and 20 mg/mL, benzoate in an amount between about 10 and 30 mg/mL, with water making up the remainder of the composition.

In a further embodiment of the first aspect, the composition is an emulsion composition intended for oral administration comprising: cholecalciferol in an amount between 10,000 IU and 100,000 IU per mL, rice bran oil in an amount between about 225 and 350 mg/mL, gum arabic in an amount between about 75 and 110 mg/mL, sorbate in an amount between about 5 and 15 mg/mL, benzoate in an amount between about 10 and 30 mg/mL, with water making up the remainder of the composition.

In a second aspect the present invention provides a method for preparing a liquid composition of the first aspect, the method comprising:
a) admixing a surfactant and water to provide a surfactant/water mixture;
b) admixing vitamin D, or a salt thereof, and an oil to provide a vitamin D/oil mixture;
c) admixing the surfactant/water mixture and the vitamin D/oil mixture;
d) adding one or more preservatives to the mixture obtained following step c); and
e) adding water to the mixture obtained following step d).

Step a) may comprise agitating the surfactant water mixture, for example by stirring. Step b) may comprise agitating the vitamin D/oil mixture, for example by stirring. Step c) may comprise admixing the vitamin D/oil mixture and the surfactant water mixture under homogenisation conditions so as to produce an emulsion. Methods for homogenising liquids are well known to those skilled in the art, and include, for example high shear mixers. Step d) may comprise adding one or more preservatives and water to the mixture obtained following step c), and agitating the resulting mixture. The method may further comprise adding one or more preservatives as part of step b) and/or step a). The method may further comprise agitating the mixture obtained following step e) until a homogeneous or substantially homogenous solution is obtained. Flavours and/or colours may also be added at any stage, for example as part of step d). Where it is desired to maximise the shelf life of the composition, the amount of vitamin D added in step b) may be about 1.5 to 2 times the desired amount. For example, if it is desired to prepare a composition comprising 10,000 IU of vitamin D, an amount of 15,000 IU of vitamin D may be added in step b).

In a third aspect the present invention provides a method for the prevention and/or treatment of a disease or condition in a subject associated with a deficiency of vitamin D, said method comprising administration to the subject of a therapeutically effective amount of a composition of the first aspect.

Diseases and conditions associated with vitamin D deficiency for which the method of the third aspect may find utility include, but are not limited to: vitamin D deficiency syndrome, rickets, osteoporosis, hypertension, chronic fatigue, chronic pain, autoimmune diseases, hyperparathyroidism, high blood pressure, tuberculosis, cancer, depression, heart disease, stroke, periodontal disease, MS, seasonal affective disorder and memory loss. It is however to be understood that the method of the third aspect is applicable to any and all diseases and/or conditions which are either directly or indirectly associated with a deficiency of vitamin D.

The method of the third aspect may form part of a combination treatment regimen for a disease or condition. For example, in the treatment or prevention of osteoporosis the vitamin D compositions of the present invention may be administered with calcium supplements and bisphosphonates. In the treatment of rickets in a child for example, the vitamin D compositions may be administered with phosphates and human growth hormone. The treatment regimen may also include exposing the child to UVB radiation for short time periods.

The method of the third aspect may also involve administering the compositions to a subject who is at risk of, or predisposed to, a disease or condition associated with vitamin D deficiency.

In a fourth aspect, the present invention provides a method for supplementing the amount of vitamin D in the body of a subject, said method comprising administration to the subject of a composition of the first aspect. A balanced level of vitamin D has long been recognised as essential to health. Accordingly, the compositions of the present invention may be used as supplements in order to assist a subject in maintaining an adequate level of vitamin D in their body. Subjects for whom the method of the fourth aspect may be useful include, but are not limited to: older people (as the body's ability to metabolise vitamin D to its active form decreases with age), people who are not regularly exposed to sunlight (for example those living at high latitudes and people who cover their skin for cultural or religious reasons), people at risk of osteoporosis and cardiovascular disease, people with diseases or conditions that impair the conversion of vitamin D to active metabolites (such as liver or kidney disease) and people with disorders that limit absorption of vitamin D from the GI tract.

The present invention will now be described with reference to specific examples, which should not be construed as in any way limiting the scope of the invention.

### EXAMPLES

### Example 1

### Compositions Comprising 10,000 IU and 50,000 IU of Vitamin D Per mL

10,000 IU composition. Amounts below are per mL (approx. 1 g) of the composition.

| Component | Amount |
|---|---|
| Cholecalciferol | 10,000 IU |
| Acacia (gum arabic) | 92 mg |
| Rice bran oil | 335 mg |
| Sodium benzoate | 14 mg |
| Potassium sorbate | 10 mg |
| Vanilla flavour (Trusil nature | 3 mg |
| identical vanilla flavour 179522 | |
| Water | Up to 1 g |

50,000 IU composition. Amounts below are per mL (approx. 1 g) of the composition.

| Component | Amount |
|---|---|
| Cholecalciferol | 50,000 IU |
| Acacia (gum arabic) | 93.1 mg |
| Rice bran oil | 258 mg |
| Sodium benzoate | 25 mg |
| Potassium sorbate | 10 mg |
| Vanilla flavour (Trusil nature identical | 3 mg |
| vanilla flavour 179522 | |
| Water | Up to 1 g |

### Example 2

### Preparation of a Composition Comprising 50,000 IU of Vitamin D Per mL

A composition in the form of a liquid emulsion comprising 50,000 IU of vitamin D per mL was prepared as follows. The method below describes preparation of 100 g (approx. 100 mL) of the composition.
1. Transfer water (19 g) into container 1, which is a Silverson in line homogeniser.
2. Activate the Silverson and slowly add acacia (Agrigum™ Spray R, (Agrisales Limited, London) 9.31 g). Continue mixing for about 30 minutes, or until the acacia is completely dissolved.
3. Mix cholecalciferol (1000 IU/mg, 5.0 g) and rice bran oil (refined, (Henry Lamotte GmbH, Bremen Germany) 25.8 g) in container 2.
4. Slowly add the mixture from container 2 to water/acacia mixture in container 1 under homogenisation conditions.
5. Transfer the mixture in container 1 (from step 4) to container 3, and wash container 1 with water (9.7 g). The water used for the wash is then transferred to container 3.
6. To container 4 is added water (6 g); sodium benzoate (BP, 2.5 g), potassium sorbate (BP, 1 g) and vanilla flavour (Trusil nature identical vanilla flavour 179522, 0.3 g). The resulting mixture is stirred.
7. Transfer the mixture in container 4 to container 3.
8. Wash container 4 with water (3 g), and transfer the water used for the wash to container 3.
9. Make up the weight of the composition in container 3 to 100 mL by adding water (15.89 g) and mix the resultant solution until homogeneous.
   The resulting composition is a clear liquid emulsion which is storage stable for a period of up to 3 years. Microbial testing of the formulation yielded the following results:

| Test | Result |
|---|---|
| Total plate count | NMT 10,000 cfu/mL |
| Yeast and mould count | NMT 100 cfu/mL |
| Enterobacteriaceae | NMT 100 cfu/mL |
| E. Coli detection | Not detected per mL |
| Salmonella spp | Not detected per 10 mL |
| Pseudomonas aeruginosa | Not detected per mL |
| Staphylococcus spp | Not detected per mL |

### Example 3

### Preparation of a Composition Comprising 10,000 IU of Vitamin D Per mL

A composition in the form of a liquid emulsion comprising 10,000 IU of vitamin D per mL was prepared as follows. The method below describes preparation of 100 g (approx. 100 mL) of the composition.
1. Transfer water (18.5 g) into container 1, which is a Silverson in line homogeniser.
2. Activate the Silverson and slowly add acacia (Agrigum™ Spray R, (Agrisales Limited, London), 9.2 g). Continue mixing for about 30 minutes, or until the acacia is completely dissolved.
3. Mix cholecalciferol (10001 U/mg, 1.0 g) and rice bran oil (refined, (Henry Lamotte GmbH, Bremen Germany), 33.5 g) in container 2.
4. Slowly add mixture from container 2 to water/acacia mixture in container 1 under homogenisation conditions.
5. Transfer the mixture in container 1 (from step 4) to container 3, and wash container 1 with water (9.7 g). The water used for the wash is then transferred to container 3.
6. To container 4 is added water (6 g), sodium benzoate (BP, 1.4 g), potassium sorbate (BP, 1.0 g) and vanilla flavour (Trusil nature identical vanilla flavour 179522, 0.3 g). The resulting mixture is stirred.
7. Transfer the mixture in container 4 to container 3.
8. Wash container 4 with water (3 g), and transfer the water used for the wash to container 3.
9. Make up the weight of the composition in container 3 to 100 mL by adding water (15.90 g) and mix the resultant solution until homogeneous.
   The resulting composition is a clear liquid emulsion which is storage stable for a period of up to 3 years. Microbial testing of the formulation yielded the following results:

| Test | Result |
|---|---|
| Total plate count | NMT 10,000 cfu/mL |
| Yeast and mould count | NMT 100 cfu/mL |
| Enterobacteriaceae | NMT 100 cfu/mL |
| E. Coli detection | Not detected per mL |
| Salmonella spp | Not detected per 10 mL |
| Pseudomonas aeruginosa | Not detected per mL |
| Staphylococcus spp | Not detected per mL |

We claim:

1. A stable liquid emulsion composition comprising vitamin D, or a salt thereof, in an amount between about 5000 IU and about 250,000 IU per mL of the stable liquid emulsion composition, sorbate and benzoate.

2. The stable liquid emulsion composition of claim 1, wherein the vitamin D, or a salt thereof, is present in an amount between about 5000 IU and about 100,000 IU per mL of the stable liquid emulsion composition.

3. The stable liquid emulsion composition of claim 1, wherein the vitamin D is present in the stable liquid emulsion composition as 1α,25-dihydroxy vitamin D₃, or an analogue or derivative thereof.

4. The stable liquid emulsion composition of claim 1, wherein the vitamin D is present in the stable liquid emulsion composition as a precursor of 1α,25-dihydroxy vitamin D₃ or as a precursor of an analogue or derivative of 1α,25-dihydroxy vitamin D₃, wherein the precursor of 1α,25-dihydroxy vitamin D₃ or the precursor of an analogue or derivative of 1α,25-dihydroxy vitamin D₃ is metabolised in vivo to provide 1α,25-dihydroxy vitamin D₃ or an analogue or derivative of 1α,25-dihydroxy vitamin D₃.

5. The stable liquid emulsion composition of claim 4, wherein the precursor is cholecalciferol.

6. The stable liquid emulsion composition of claim 1, wherein the vitamin D is present in the stable liquid emulsion composition as ergocalciferol, or a metabolite, analogue or derivative thereof.

7. The stable liquid emulsion composition of claim 1, further comprising an edible oil.

8. The stable liquid emulsion composition of claim 7, wherein the edible oil is a vegetable oil.

9. The stable liquid emulsion composition of claim 8, wherein the vegetable oil is rice bran oil.

10. The stable liquid emulsion composition of claim 1, further comprising a surfactant.

11. The stable liquid emulsion composition of claim 10, wherein the surfactant is a natural gum or a cellulosic gum.

12. The stable liquid emulsion composition of claim 1, wherein the ratio by mass of the amount of benzoate present in the stable liquid emulsion composition to the amount of sorbate present in the stable liquid emulsion composition is between about 1:1 and about 5:1.

13. The stable liquid emulsion composition of claim 1, wherein the sorbate is potassium sorbate.

14. The stable liquid emulsion composition of claim 1, wherein the benzoate is sodium benzoate.

15. The stable liquid emulsion composition of claim 1, wherein the emulsion is an oil-in-water emulsion or a water-in-oil emulsion.

16. The stable liquid emulsion composition of claim 1, further comprising flavours and/or colours.

17. The stable liquid emulsion composition of claim 1, which is intended for oral administration.

18. A stable liquid emulsion composition comprising: vitamin D in an amount between about 10,000 IU and 250,000 IU per mL, water, an edible oil, a natural gum, sorbate and benzoate.

19. The stable liquid emulsion composition of claim 18, wherein the edible oil is present in an amount between about 200 and 400 mg/mL, the natural gum is present in an amount between about 60 and 120 mg/mL, the sorbate is present in an amount between about 5 and 70 mg/mL, the benzoate is present in an amount between about 10 and 140 mg/mL, with water making up the remainder of the stable liquid emulsion composition.

20. The stable liquid emulsion composition of claim 19, wherein the edible oil is rice bran oil.

21. The stable liquid emulsion composition of claim 20, wherein the natural gum is gum arabic.

22. A method for preparing a stable liquid emulsion composition as defined claim 1, the method comprising:
a) admixing a surfactant and water to provide a surfactant/water mixture;
b) admixing vitamin D, or a salt thereof, and an oil to provide a vitamin D/oil mixture;
c) admixing the surfactant/water mixture and the vitamin D/oil mixture;
d) adding sorbate and benzoate to the mixture obtained following step c); and
e) adding water to the mixture obtained following step d).

23. The method of claim 22, comprising agitating the surfactant/water mixture.

24. The method of claim 22, wherein step b) comprises agitating the vitamin D/oil mixture.

25. The method of claim 22, wherein step c) comprises admixing the vitamin D/oil mixture and the surfactant/water mixture under homogenisation conditions.

26. The method of claim 22, wherein step d) comprises adding sorbate, benzoate and water to the mixture obtained following step c).

27. The method of claim 22, further comprising agitating the mixture obtained following step e) until a homogeneous liquid is obtained.

28. The method claim 22, further comprising adding sorbate, benzoate as part of step b).

29. A method for the prevention and/or treatment of a disease or condition in a subject associated with a deficiency of vitamin D, said method comprising administration to the subject of a therapeutically effective amount of a stable liquid emulsion composition of claim 1.

30. The method of claim 29, wherein the disease or condition is selected from the group consisting of: vitamin D deficiency syndrome, rickets, osteoporosis, hypertension, chronic fatigue, chronic pain, autoimmune diseases, hyperparathyroidism, high blood pressure, tuberculosis, cancer, depression, heart disease, stroke, periodontal disease, MS, seasonal affective disorder and memory loss.

31. A method for supplementing the amount of vitamin D in the body of a subject, said method comprising administration to the subject of a stable liquid emulsion composition according to claim 1.

32. The stable liquid emulsion composition of claim 2, wherein the vitamin D, or a salt thereof, is present in an amount between about 20,000 IU and 100,000 IU per mL of the stable liquid emulsion composition.

33. The stable liquid emulsion composition of claim 2, wherein the vitamin D, or a salt thereof, is present in an amount between about 10,000 IU and about 50,000 IU per mL of the stable liquid emulsion composition.
